(19) United States
(12) Patent Application Publication
(10) Pub. No.: US 2021/0047192 A1
(43) Pub. Date: Feb. 18, 2021

(54) DELAYED RELEASE FORMULATION OF NITRIFICATION INHIBITORS

(71) Applicant: BASF SE, Ludwigshafen (DE)

(72) Inventors: Barbara Nave, Limburgerhoff (DE); Karl-Heinrich Schneider, Limburgerhof (DE); Ulrich Mueller, Ludwigshafen (DE); Lukasz Karwacki, Ludwigshafen (DE); Lena Arnold, Antwerpen (BE); Anderi-Nicolae Parvulescu, Ludwigshafen (DE)

(21) Appl. No.: 16/976,608

(22) PCT Filed: Mar. 6, 2019
(86) PCT No.: PCT/EP2019/055483

§ 371 Date: Aug. 28, 2020

(30) Foreign Application Priority Data
Mar. 12, 2018 (EP) 18161231.8

(51) Int. Cl. C01B 39/02; C05G 3/90; C05D 9/02; C05C 3/00; C07C 43/215; C07C 43/176; C07D 249/08; C07C 235/42; C07C 233/65; B01J 20/30; C05G 5/12

(52) U.S. Cl. CPC C01B 39/026; C05G 3/90; C05D 9/02; C05C 3/00; C07C 43/215; C05G 5/12; C07D 249/08; C07C 235/42; C07C 233/65; B01J 20/3028; C07C 43/176

(57) ABSTRACT

The invention relates to a composition comprising a) zeolitic imidazolate framework ZIF-8; and b) Compounds of formula (I) or a stereoisomer, salt, tautomer or N-oxide thereof, wherein the variables have a meaning as defined in the main body of the text. It also relates to a method for fertilization comprising treatment with the composition. Other objects are the use of ZIF-8 for reducing the evaporation rate of Compounds of formula (I); a method for production of the composition as defined comprising step a) of adsorbing Compounds of formula (I) on ZIF-8; and the use of the composition for producing granules comprising Compounds of formula (I) and a fertilizer.

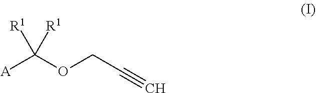

The invention relates to a composition comprising the zeolitic imidazolate framework ZIF-8 and Compounds of formula (I)

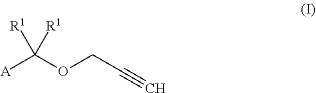

or a stereoisomer, salt, tautomer or N-oxide thereof, wherein the variables have the following meaning:
R¹, R² independently H;
C₁-C₆-alkyl, C₂-C₆-alkenyl, C₂-C₆-alkynyl, C₁-C₆-haloalkyl, C₁-C₄-alkoxy-C₁-C₄-alkyl C₁-C₆-alkoxy, C₂-C₆-alkenyloxy, C₂-C₆-alkynyloxy, which groups are unsubstituted or substituted by one or more, same or different R^{e}; or
C₃-C₈-cycloalkyl, C₃-C₈-cycloalkenyl, heterocyclyl, aryl, hetaryl, C₃-C₈-cycloalkyl-C₁-C₆-alkyl, C₃-C₈-cycloalkenyl-C₁-C₆-alkyl, heterocyclyl-C₁-C₆-alkyl, aryl-C₁-C₆-alkyl, hetaryl-C₁-C₆-alkyl, phenoxy, or benzyloxy, wherein the cyclic moieties are unsubstituted or substituted by one or more, same or different R^{a};
   A phenyl, which is unsubstituted or substituted with one or more, same or different R^{A};
R^{A} CN, halogen, NO₂, OR^{b}, NR^{c}R^{d}, C(Y)R^{b}, C(Y)OR^{b}, C(Y)NR^{c}R^{d}, S(Y)ₘR^{b}, S(Y)ₘOR^{b}; C₁-C₆-alkyl, C₂-C₆-alkenyl, C₂-C₆-alkynyl, C₁-C₆-haloalkyl, C₁-C₆-alkoxy, C₁-C₆-alkylthio, which groups are unsubstituted or substituted by one or more, same or different R^{e}; or
   C₃-C₈-cycloalkyl, C₃-C₈-cycloalkenyl, heterocyclyl, aryl, hetaryl, C₃-C₈-cycloalkyl-C₁-C₆-alkyl, C₃-C₈-cycloalkenyl-C₁-C₆-alkyl, heterocyclyl-C₁-C₆-alkyl, aryl-C₁-C₆-alkyl, hetaryl-C₁-C₆-alkyl, phenoxy, benzyloxy, wherein the cyclic moieties are unsubstituted or substituted by one or more, same or different R^{a};
   R^{a} CN, halogen, NO₂, C₁-C₄-alkyl, C₁-C₄-haloalkyl, or C₁-C₄-alkoxy;
      or two substituents R^{a} on adjacent C-atoms form a bridge CH₂CH₂CH₂CH₂, OCH₂CH₂CH₂, CH₂OCH₂CH₂, OCH₂CH₂O, OCH₂OCH₂, CH₂CH₂CH₂, CH₂CH₂O, CH₂OCH₂, O(CH₂)O, SCH₂CH₂CH₂, CH₂SCH₂CH₂, SCH₂CH₂S, SCH₂SCH₂, CH₂CH₂S, CH₂SCH₂, S(CH₂)S, and form together with the C atoms, to which the two R^{a} are bonded to, a 5-membered or 6-membered saturated carbocyclic or heteocyclic ring;
      R^{b} H, C₁-C₆-alkyl, C₂-C₄-alkenyl, C₂-C₄-alkynyl, C₁-C₄-haloalkyl, phenyl or benzyl;
      R^{c}, R^{d} are independently of each other H, C₁-C₄-alkyl, or C₁-C₄-haloalkyl; or
         R^{c} and R^{d} together with the N atom to which they are bonded form a 5- or 6-membered, saturated or unsaturated heterocycle, wherein the heterocycle is unsubstituted or substituted with one or more, same or different halogen atoms;
      R^{e} CN, halogen, C₁-C₄-alkyl, C₁-C₄-haloalkyl, C₁-C₄-alkoxy, or C₁-C₄-haloalkoxy;

### Y O or S; and

m 0, 1 or 2.

The invention also relates to a method for reducing nitrification, comprising treating plant propagation material, a plant growing on soil or soil substituents and/or the locus or soil or soil substituents where the plant is growing or is intended to grow with the composition; and to the use of the composition for reducing nitrification. It also relates to a method of fertilization, comprising treating plant propagation material, a plant growing on soil or soil substituents and/or the locus or soil or soil substituents where the plant is growing or is intended to grow with the composition; and to the use of the composition for reducing nitrification. It also relates to the use of ZIF-8 for reducing the evaporation rate of Compounds of formula (I). Another object is a method for production of the composition comprising the step of adsorbing Compounds of formula (I) on ZIF-8; and seeds comprising the composition. Combinations of embodiments with other embodiments are within the scope of the invention.

Fertilization technology is of growing impact due to the ever-increasing population of the world on the one hand side and the limited agriculturally available area on the other. Nearly 90% of the nitrogen fertilizers applied worldwide are in the NH₄⁺-form (Subbarao et al., 2012, Advances in Agronomy, 114, 249-302). This is, inter alia, due to the fact that NH₄⁺-assimilation is energetically more effective than assimilation of other nitrogen sources, such as of NO₃⁻-containing fertilizers. Another advantage of NH₄⁺ as compared to NO₃ is that it has a longer residence time in the soil. This effect is due to higher leaching of NO₃⁻, and to the removal of NO₃ by microbiological conversion to gaseous nitrogen-containing molecules, such as N₂O and N₂. It is thus desirable to maintain the reduced NH₄⁺-form in the soil for as long as possible. However, NH₄⁺ is converted by soil microorganisms to NO₃⁻ in a relatively short time in a process known as nitrification. The nitrification is carried out primarily by chemolithographic bacteria, effecting an enzymatic transformation by virtue of ammonia monooxigenase. Nitrification typically leads to nitrogen leakage and environmental pollution. Approximately 50% of the applied nitrogen fertilizers are lost during the year following fertilizer addition (Nelson et Huber, Nitrification inhibitors for corn production, 2001, National Corn Handbook, Iowa State University). As a countermeasure against the loss by nitrification, nitrification inhibitors are commonly used. Compounds of formula (I) are especially useful nitrification inhibitors known from WO2016/075289 with a high efficacy, and a good environmental safety.

A disadvantage of Compounds of formula (I), however, is the high evaporation rate. This drawback is particularly pronounced if Compounds of formula (I) are applied by spraying and/or at elevated temperatures, e.g. at temperatures above 30° C. This reduces their efficacy for the farmer with regard net bioavailability at target, their long-term effect in the soil, and curtails the available application methods. The high evaporation rate of Compounds of formula (I) is also detrimental for production processes in which Compounds of formula (I) are processed at elevated temperatures, especially in granulation processes. It was hence an object of the present invention to find a composition comprising Compounds of formula (I) with a reduced evaporation rate. It was also an object to find uses and methods of application for reducing the evaporation rate of Compounds of formula (I), and for reducing the evaporation rate of Compounds of formula (I) in compositions. Another object was to find methods of production for compositions comprising Compounds of formula (I) that may be carried out at elevated temperatures, such as temperatures of from 50° C. to 150° C.

These objects were successfully addressed by a composition comprising Compounds of formula (I) and the zeolitic imidazolate framework ZIF-8; by uses and methods of application of ZIF-8 for reducing the evaporation rate of Compounds of formula (I); and by a method of production of compositions comprising Compounds of formula (I), especially wherein the compositions comprise fertilizers, comprising the step of adsorbing Compounds of formula (I) on ZIF-8.

Further advantages of the composition are that the means for reducing the evaporation rate of Compounds of formula (I), ZIF-8, is biodegradable, and does not require a desiccation step prior to adsorption of Compounds of formula (I).

The composition comprises the zeolithic imidazolate framework ZIF-8 belonging to the class of metal-organic frameworks (MOF). MOFs are highly porous crystalline materials comprising metal ions or clusters coordinated by organic ligands, forming one-, two-, or three-dimensional structures. ZIF-8 is commercially available, e.g. under the tradename Basolite® Z1200, and can be produced as described in Tanaka S. et al., Chem. Let. 2012, vol. 41, pp 1337-1339, or Shi Q., Angew. Chem. Int. Ed., 2011, vol. 50, pp 672-675, or WO2013/005160A1 (Examples, p. 12-15).

ZIF-8 comprises Zn²⁺-ions, which are coordinated by methyl imidazole ligands. ZIF-8 is characterized by a pore size of approximately 11.6 Å and a BET surface area of ca. 1600 m²/g (Tanaka S. et al., Chem. Let. 2012, vol. 41, pp 1337-1339). It has surprisingly been found that ZIF-8 is particularly useful for achieving the objects described above compared to other MOFs or zeolites with comparable characteristics, e.g. similar surface areas or pore sizes.

The weight ratio of Compounds of formula (I) to ZIF-8 in the composition is usually 10:1 to 1:100, preferably 2:1 to 1:50, more preferably 1:3 to 1:50, and in particular 1:3 to 1:10. The weight ratio of Compounds of formula (I) to ZIF-8 may be at least 1:30, preferably at least 1:20. The weight ratio of Compounds of formula (I) to ZIF-8 may be up to 5:1, preferably up to 1:3, more preferably up to 1:4.

The composition may comprise from 1 to 90 wt % of Compounds of formula (I), preferably 1 to 60 wt %, more preferably 1 to 30 wt % with regard to the total weight of the composition. The composition usually comprises at least 0.5 wt % of Compounds of formula (I), more preferably at least 1 wt %, most preferably at least 2 wt %, especially preferably at least 10 wt %, and particularly preferably at least 20 wt % with regard to the total weight of the composition. The composition may contain up to 80 wt %, preferably up to 70 wt %, more preferably up to 40 wt %, most preferably up to 10 wt % of Compounds of formula (I) with regard to the total weight of the composition.

ZIF-8 is solid at 25° C. and may be utilized in the composition, as well as the uses and methods of the invention in the form of a powder, or in the form of granules.

The variables of Compounds of formula (I) may have the following meanings:

R^{a} may be halogen, C₁-C₂-alkyl, C₁-C₂-alkoxy; or two substituents R^{a} on adjacent C-atoms may be a OCH₂CH₂O bridge or a O(CH₂)O bridge.

R^{b} may be H, C₁-C₆-alkyl, phenyl or benzyl.

R^{c} and R^{d} may be independently H, C₁-C₄-alkyl, or C₁-C₄-haloalkyl.

R^{e} may be halogen, or C₁-C₄-alkyl.

R^{A} may be halogen, NO₂, NR^{c}R^{d}, C₁-C₆-alkyl, C₁-C₆-haloalkyl, C₁-C₆-alkoxy, C₁-C₆-alkylthio, phenoxy or benzyloxy, wherein the cyclic moieties may be unsubstituted or substituted with one or more, same or different R^{a}. Preferably R^{A} is halogen, more preferably Cl.

R¹ and R² may be independently H, C₂-C₆-alkynyl, C₂-C₆-alkynyloxy, aryl-C₁-C₆-alkyl, or hetaryl-C₁-C₆-alkyl, wherein preferably at least one of R¹ and R² is H. Typically, R¹ and R² are H.

A may be phenyl, which is unsubstituted or substituted with 1, 2, or 3, same or different R^{A}, preferably substituted with 1 or 2, same or different R^{A}, most preferably substituted with one R^{A}.

In one embodiment, R¹ is H and R² is C₂-C₆-alkynyl, C₂-C₆-alkynyloxy, aryl-C₁-C₆-alkyl, or hetaryl-C₁-C₆-alkyl; preferably C₂-C₄-alkynyl, C₂-C₄-alkynyloxy, aryl-C₁-C₄-alkyl, or hetaryl-C₁-C₄-alkyl; most preferably hetaryl-C₁-C₄-alkyl; and in particular triazolylmethyl.

These compounds correspond to Compounds of formula (Ia).

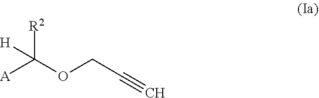

If R² is triazolylmethyl, it is preferred that the triazole moiety is bonded to the methyl group via one of the nitrogen atoms. Furthermore, it is preferred that the triazole moiety is a 1,2,4-triazole moiety.

In another preferred embodiment, both R¹ and R² are H. These compounds correspond to Compounds of formula (Ib).

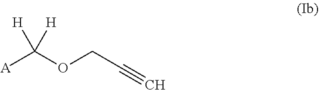

In another embodiment, A is phenyl, which is unsubstituted or substituted with one or more, same or different R^{A}. Such compounds correspond to Compounds of formula (Ic), wherein n is 0, 1, 2, 3, 4, or 5. Usually, the variable n is 1 or 2.

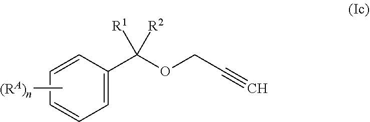

In connection with the compounds defined above, it is to be understood that the substituent(s) R^{A} may be present at any carbon atom of the phenyl ring.

In one embodiment, the present invention relates to Compounds of formula (I), wherein R¹ is H, A is phenyl, which is unsubstituted or substituted with one or more, same or different R^{A}, and wherein R² is C₂-C₆-alkynyl, C₂-C₆-alkynyloxy, aryl-C₁-C₆-alkyl, or hetaryl-C₁-C₆-alkyl, preferably C₂-C₄-alkynyl, C₂-C₄-alkynyloxy, aryl-C₁-C₄-alkyl, or hetaryl-C₁-C₄-alkyl, more preferably C₃-alkynyloxy or hetaryl-C₁-C₄-alkyl, most preferably hetaryl-C₁-C₄-alkyl, in particular triazolylmethyl.

In another embodiment, the present invention relates to Compounds of formula (I), wherein R¹ is H, R² is H, A is phenyl, which is unsubstituted or substituted with one or more, same or different R^{A}. Such compounds are Compounds of formula (Id).

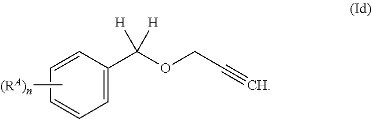

In one embodiment, n in Compounds of formula (Id) is 0. In another embodiment, n in Compounds of formula (Id) is 1. In another embodiment, n in Compounds of formula (Id) is 2.

In connection with the compounds defined above, it is to be understood that the substituent(s) R^{A} may be present at any carbon atom of the phenyl ring. In certain preferred embodiments of the invention, it is preferred that at least one substituent R^{A} is present in para position with respect to the propargylether group.

In one embodiment, R^{A} is halogen, NO₂, NR^{c}R^{d}, C₁-C₆-alkyl, C₁-C₆-haloalkyl, C₁-C₆-alkoxy, C₁-C₆-alkylthio, phenoxy, or benzyloxy, wherein the cyclic moieties are unsubstituted or substituted with one or more, same or different R^{a}; wherein

R^{a} is halogen, C₁-C₂-alkyl, C₁-C₂-alkoxy, or two substituents R^{a} on adjacent C-atoms are a OCH₂CH₂O bridge or a O(CH₂)O bridge; and

R^{c} and R^{d} are independently of each other H, C₁-C₄-alkyl, or C₁-C₄-haloalkyl.

In another embodiment, R^{A} is halogen, NO₂, C₁-C₆-alkyl, C₁-C₆-haloalkyl, C₁-C₆-alkoxy, or phenoxy, wherein the phenoxy group is unsubstituted or substituted with one or more, same or different halogen atoms.

In another embodiment, R^{A} is halogen, NO₂, C₁-C₂-alkyl, C₁-C₂-haloalkyl, C₁-C₂-alkoxy, or phenoxy, wherein the phenoxy group may be unsubstituted or substituted with one or more, same or different halogen atoms.

In another embodiment, R^{A} is F, C, Br, NO₂, CH₃, CF₃, methoxy, or phenoxy, wherein the phenoxy group is unsubstituted or substituted with one or more, same or different halogen atoms selected from F, Cl, and Br.

In another embodiment, in particular in case of Compounds of formula (Id) as defined above, R^{A} is halogen, C₁-C₄-alkyl, and C₁-C₄-alkoxy. In another embodiment, in particular in case of Compounds of formula (Id) as defined above, R^{A} is F, Cl, Br, I, CH₃, methoxy, ethoxy, and n-propoxy, wherein preferably at least one of these groups is present in para position with respect to the propargylether group.

In one embodiment, the variables of Compounds of formula (I) are defined as follows:

R¹, R² independently H; or
C₂-C₆-alkynyl, C₂-C₆-alkynyloxy, aryl-C₁-C₆-alkyl, or hetaryl-C₁-C₆-alkyl, provided at least one of R¹ and R² is H;
   A phenyl, which is unsubstituted or substituted with one or more, same or different R^{A};
R^{A} CN, halogen, NO₂, C(Y)OR^{b}, C(Y)NR^{c}R^{d}, NR^{c}R^{d}, C₁-C₆-alkyl, C₁-C₆-haloalkyl, C₁-C₆-alkoxy, C₂-C₆-alkynyloxy, C₁-C₆-alkylthio; or
   phenoxy, benzyloxy, wherein the cyclic moieties are unsubstituted or substituted with one or more, same or different R^{a};
   R^{a} halogen, C₁-C₂-alkyl, C₁-C₂-alkoxy, or two substituents R^{a} on adjacent C-atoms are a OCH₂CH₂O bridge, or a O(CH₂)O bridge;
   R^{b} H or C₁-C₄-alkyl;
   R^{c}, R^{d} are independently H, C₁-C₄-alkyl, or C₁-C₄-haloalkyl.

In another embodiment, the variables of Compounds of formula (I) are defined as follows:

R¹, R² independently H; or
C₂-C₆-alkynyl, C₂-C₆-alkynyloxy, aryl-C₁-C₆-alkyl, or hetaryl-C₁-C₆-alkyl, provided at least one of R¹ and R² is H;
   A phenyl, which is unsubstituted or substituted with one or more, same or different R^{A};
R^{A} halogen, NO₂, NR^{c}R^{d}, C₁-C₆-alkyl, C₁-C₆-haloalkyl, C₁-C₆-alkoxy, C₁-C₆-alkylthio; or phenoxy, benzyloxy, wherein the cyclic moieties are unsubstituted or substituted with one or more, same or different R^{a};
   R^{a} halogen, C₁-C₂-alkyl, C₁-C₂-alkoxy, or two substituents R^{a} on adjacent C-atoms are a OCH₂CH₂O bridge, or a O(CH₂)O bridge;
   R^{c}, R^{d} are independently H, C₁-C₄-alkyl, or C₁-C₄-haloalkyl.

In another embodiment, the variables of Compounds of formula (I) are defined as follows:
R¹ H;
R² C₂-C₆-alkynyl, C₂-C₆-alkynyloxy, aryl-C₁-C₆-alkyl, or hetaryl-C₁-C₆-alkyl; preferably C₂-C₄-alkynyl, C₂-C₄-alkynyloxy, aryl-C₁-C₄-alkyl, or hetaryl-C₁-C₄-alkyl; more preferably C₃-alkynyloxy or hetaryl-C₁-C₄-alkyl; most preferably hetaryl-C₁-C₄-alkyl; in particular triazolylmethyl;
A phenyl, which is unsubstituted or substituted with one or more, same or different R^{A};
   R^{A} halogen, NO₂, C₁-C₆-alkyl, C₁-C₆-haloalkyl, C₁-C₆-alkoxy; or phenoxy, which is unsubstituted or substituted with one or more, same or different halogen atoms.

In another embodiment, the variables of Compounds of formula (I) are defined as follows:
R¹ H;
R² C₂-C₆-alkynyl, C₂-C₆-alkynyloxy, aryl-C₁-C₆-alkyl, or hetaryl-C₁-C₆-alkyl; preferably C₂-C₄-alkynyl, C₂-C₄-alkynyloxy, aryl-C₁-C₄-alkyl, or hetaryl-C₁-C₄-alkyl; more preferably C₃-alkynyloxy or hetaryl-C₁-C₄-alkyl; most preferably hetaryl-C₁-C₄-alkyl; in particular triazolylmethyl;
A phenyl, which is unsubstituted or substituted with one or more, same or different R^{A};
   R^{A} F, Cl, Br, NO₂, CH₃, CF₃, CH₃O, phenoxy, which is unsubstituted or substituted by one or more, same or different halogen atoms selected from F, Cl, and Br.

In one embodiment, the variables of Compounds of formula (Id) are defined as follows:
R^{A} CN, halogen, NO₂, C(Y)OR^{b}, C(Y)NR^{c}R^{d}, C₁-C₆-alkyl, C₁-C₆-haloalkyl, C₁-C₆-alkoxy, C₂-C₆-alkynyloxy, or phenoxy, which is unsubstituted or substituted with one or more, same or different R^{a};
   R^{a} halogen, C₁-C₂-alkyl, or C₁-C₂-alkoxy;
   R^{b} H, or C₁-C₄-alkyl;
   R^{c}, R^{d} are independently H, or C₁-C₄-alkyl.

In another embodiment, the Compounds of formula (I) relate to Compounds of formula (Id) and R^{A} is halogen, NO₂, C₁-C₆-alkyl, C₁-C₆-haloalkyl, C₁-C₆-alkoxy, or phenoxy, which is unsubstituted or substituted with one or more, same or different halogen atoms.

In another embodiment, the Compounds of formula (I) relate to Compounds of formula (Id) and R^{A} is halogen, NO₂, C₁-C₂-alkyl, C₁-C₂-haloalkyl, C₁-C₂-alkoxy, or phenoxy, which is unsubstituted or substituted with one or more, same or different halogen atoms.

In another embodiment, the Compounds of formula (I)relate to Compounds of formula (Id) and R^{A} is F, Cl, Br, NO₂, CH₃, CF₃, CH₃O, or phenoxy, which is unsubstituted or substituted with one or more, same or different halogen atoms selected from F, Cl, and Br.

In another embodiment, the Compounds of formula (I)relate to Compounds of formula (Id) and R^{A} is F, Cl, Br, NO₂, CH₃C₃, CH₃O, CH₃CH₂O, or CH₃CH₂CH₂O, wherein preferably at least one of these groups is present in para position with respect to the propargylether group.

Particularly preferred Compounds of formula (I) are compiled in Table A below.

TABLE A

| No. | Structure |
|---|---|
| 1-1 | 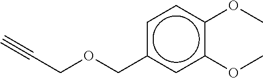 |
| 1-2 | 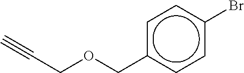 |
| 1-3 | 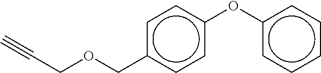 |
| 1-4 | 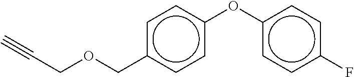 |
| 1-5 | 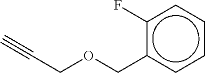 |
| 1-6 | 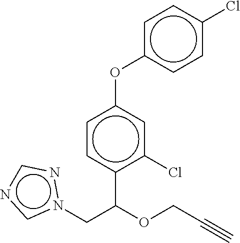 |
| 1-7 | 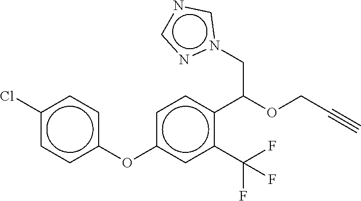 |
| 1-8 | 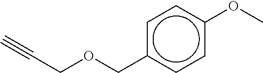 |
| 1-9 | 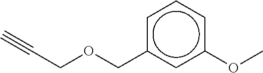 |
| 1-10 | 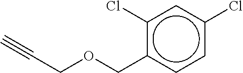 |
| 1-11 | 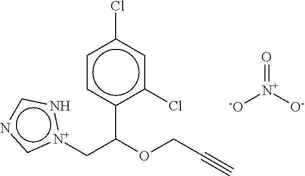 |
| 1-12 | 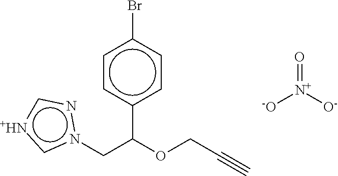 |
| 1-13 | 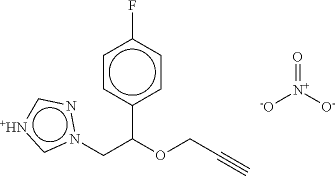 |
| 1-14 | 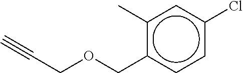 |
| 1-15 | 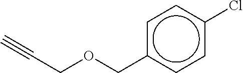 |
| 1-16 | 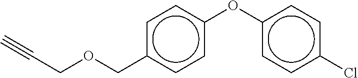 |
| 1-17 | 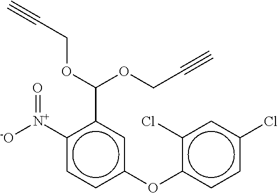 |
| 1-18 | 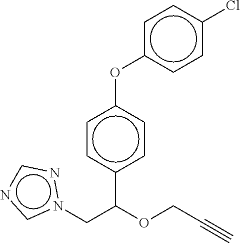 |
| 1-19 | 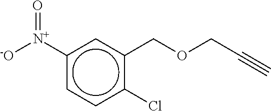 |
| 1-20 | 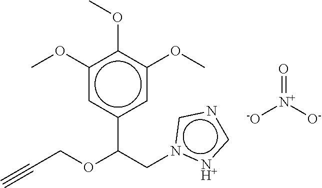 |
| 1-21 | 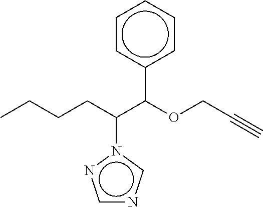 |
| 1-22 | 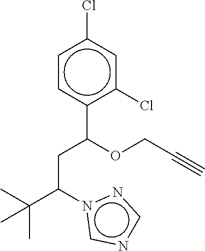 |
| 1-23 | 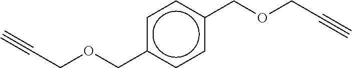 |
| 1-24 | 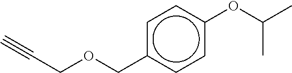 |
| 1-25 | 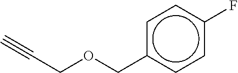 |
| 1-26 | 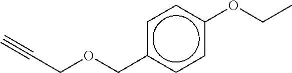 |
| 1-27 | 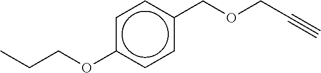 |
| 1-28 | 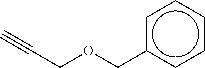 |
| 1-29 | 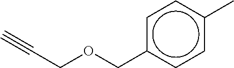 |
| 1-30 | 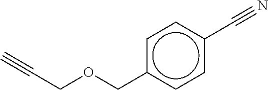 |
| 1-31 | 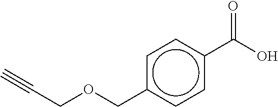 |
| 1-32 | 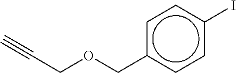 |
| 1-33 | 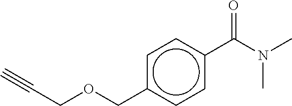 |

The compounds of Table A may be subdivided into Compounds of formula (Ia), i.e. compounds 1-6, 1-7, 1-11, 1-12, 1-13, 1-17, 1-18, 1-20, 1-21, 1-22, and Compounds of formula (Ib), i.e. compounds 1-1, 1-2, 1-3, 1-4, 1-5, 1-8, 1-9, 1-10, 1-14, 1-15, 1-16, 1-19, 1-23, 1-24, 1-25, 1-26, 1-27, 1-28, 1-29, 1-30, 1-31, 1-32, 1-33.

In one embodiment of the invention, the Compounds of formula (I) are Compounds of formula (Ia), which are selected from compounds 1-6, 1-7, 1-11, 1-12, 1-13, 1-17, 1-18, 1-20, 1-21, and 1-22, or which are structurally different from these compounds, but are characterized in that R^{A}, if present, is selected from the group consisting of F, C, Br, NO₂, CH₃, CF₃, methoxy, and phenoxy, wherein the phenoxy group is unsubstituted or substituted with 1 or 2, same or different halogen atoms selected from F, Cl, and Br.

In one embodiment, the Compounds of formula (I) are compounds 1-6, 1-7, 1-11, 1-12, 1-13, 1-17, 1-18, 1-20, 1-21, or 1-22. In another embodiment, the Compounds of formula (I) are compounds 1-1, 1-2, 1-3, 1-4, 1-5, 1-8, 1-9, 1-10, 1-14, 1-15 1-16, or 1-19, or which are structurally different from these compounds, but are characterized in that R^{A}, if present, is selected from F, Cl, Br, NO₂, CH₃, CF₃, CH₃O, and phenoxy, wherein the phenoxy group is unsubstituted or substituted with 1 or 2, same or different halogen atoms selected from F, Cl, and Br.

In another embodiment of the invention, the Compounds of formula (I) are compounds 1-1, 1-2, 1-3, 1-4, 1-5, 1-8, 1-9, 1-10, 1-14, 1-15 1-16, 1-19, 1-23, 1-24, 1-25, 1-26, 1-27, 1-28, 1-29, 1-30, 1-31, 1-32, or 1-33, or which are structurally different from these compounds, but are characterized in that R^{A}, if present, is selected from halogen, C₁-C₄-alkyl, and C₁-C₄-alkoxy, and preferably from F, C, Br, I, CH₃, CH₃O, CH₃CH₂O, and CH₃CH₂CH₂O.

In a preferred embodiment of the invention, Compounds of formula (I) are compounds 1-1, 1-2, 1-3, 1-4, 1-5, 1-8, 1-9, 1-10, 1-14, 1-15, 1-16, 1-19, 1-23, 1-24, 1-25, 1-26, 1-27, 1-28, 1-29, 1-30, 1-31, 1-32, or 1-33. In another embodiment, Compounds of formula (I) are compounds 1-2, 1-5, 1-8, 1-14, 1-15, 1-21, 1-23, 1-24, 1-25, 1-26, 1-27, 1-28, 1-29, 1-30, 1-31, 1-32, or 1-33, preferably compounds 1-8, 1-14, 1-15, 1-25, 1-26, 1-27, 1-28, 1-29, or 1-32.

In one embodiment Compound of formula (I) is the compound of formula 1-1 as defined in Table A above. In one embodiment Compound of formula (I) is the compound of formula 1-2 as defined in Table A above. In one embodiment Compound of formula (I) is the compound of formula 1-3 as defined in Table A above. In one embodiment Compound of formula (I) is the compound of formula 1-4 as defined in Table A above. In one embodiment Compound of formula (I) is the compound of formula 1-5 as defined in Table A above. In one embodiment Compound of formula (I) is the compound of formula 1-6 as defined in Table A above. In one embodiment Compound of formula (I) is the compound of formula 1-7 as defined in Table A above. In one embodiment Compound of formula (I) is the compound of formula 1-8 as defined in Table A above. In one embodiment Compound of formula (I) is the compound of formula 1-9 as defined in Table A above. In one embodiment Compound of formula (I) is the compound of formula 1-10 as defined in Table A above. In one embodiment Compound of formula (I) is the compound of formula 1-11 as defined in Table A above. In one embodiment Compound of formula (1) is the compound of formula 1-12 as defined in Table A above. In one embodiment Compound of formula (I) is the compound of formula 1-13 as defined in Table A above. In one embodiment Compound of formula (I) is the compound of formula 1-14 as defined in Table A above. In one embodiment Compound of formula (I) is the compound of formula 1-15 as defined in Table A above. In one embodiment Compound of formula (I) is the compound of formula 1-16 as defined in Table A above. In one embodiment Compound of formula (I) is the compound of formula 1-17 as defined in Table A above. In one embodiment Compound of formula (I) is the compound of formula 1-18 as defined in Table A above. In one embodiment Compound of formula (1) is the compound of formula 1-19 as defined in Table A above. In one embodiment Compound of formula (I) is the compound of formula 1-20 as defined in Table A above. In one embodiment Compound of formula (I) is the compound of formula 1-21 as defined in Table A above. In one embodiment Compound of formula (I) is the compound of formula 1-22 as defined in Table A above. In one embodiment Compound of formula (I) is the compound of formula 1-23 as defined in Table A above. In one embodiment Compound of formula (I) is the compound of formula 1-24 as defined in Table A above. In one embodiment Compound of formula (I) is the compound of formula 1-25 as defined in Table A above. In one embodiment Compound of formula (1) is the compound of formula 1-26 as defined in Table A above. In one embodiment Compound of formula (I) is the compound of formula 1-27 as defined in Table A above. In one embodiment Compound of formula (I) is the compound of formula 1-28 as defined in Table A above. In one embodiment Compound of formula (I) is the compound of formula 1-29 as defined in Table A above. In one embodiment Compound of formula (I) is the compound of formula 1-30 as defined in Table A above. In one embodiment Compound of formula (I) is the compound of formula 1-31 as defined in Table A above. In one embodiment Compound of formula (I) is the compound of formula 1-32 as defined in Table A above. In one embodiment Compound of formula (1) is the compound of formula 1-33 as defined in Table A above.

The term “nitrification inhibitor” is to be understood in the context of this specification as a chemical substance which slows down or stops the nitrification process. Nitrification inhibitors accordingly retard the natural transformation of ammonium into nitrate, by inhibiting the activity of bacteria such as Nitrosomonas spp. The term “nitrification” as used herein is to be understood as the biological oxidation of ammonia (NH₃) or ammonium (NH₄⁺) with oxygen into nitrite (NO₂⁻) followed by the oxidation of these nitrites into nitrates (NO₃⁻) by microorganisms. Besides nitrate (NO₃⁻) nitrous oxide is also produced though nitrification. Nitrification is an important step in the nitrogen cycle in soil. The inhibition of nitrification may thus also reduce N₂O losses. The term nitrification inhibitor is considered equivalent to the use of such a compound for inhibiting nitrification. Typically, the composition reduces the nitrification of at least 20% at 15° C. over a period of 3 weeks, preferably at least 40%, more preferably at least 60%, most preferably at least 80% as compared to a situation in which only Coumpounds of formula (I) are applied.

The term “Compounds of formula (I)”, “Compounds of formula (Ia)”, “Compounds of formula (Ib)”, “Compounds of formula (Ic)”, or “Compounds of formula (Id)” comprises the compound(s) as defined herein as well as a stereoisomer, salt, tautomer or N-oxide thereof, preferably the compound(s) as defined herein as well as a stereoisomer, salt, or N-oxide thereof, more preferably the compound(s) as defined herein as well as a stereoisomer or salt thereof.

It is of course to be understood that tautomers can only be present, if a substituent is present at the respective compounds, which covers tautomers such as keto-enol tautomers, imine-enamine tautomers, amide-imidic acid tautomers or the like. Furthermore, it is to be understood that stereoisomers are only possible, if there is at least one centre of chirality in the molecule or if geometrical isomers (cis/trans isomers) can be formed.

Compounds of formula (I) may be amorphous or may exist in one or more different crystalline states (polymorphs) which may have different macroscopic properties such as stability or show different biological properties such as activities. The present invention relates to compositions comprising amorphous and crystalline Compounds of formula (I), mixtures of different crystalline states of the respective compound of formula (I), as well as amorphous or crystalline salts thereof. Usually, Compounds of formula (I) utilized for the composition, the uses and methods of application, and the methods of production, are usually liquids. In case they are solids, they are usually applied in the form of a solution in water or an organic solvent, e.g. CH₃OH.

Salts of Compounds of formula (I) are preferably agriculturally acceptable salts. They can be formed in a customary manner, e.g. by reacting the compound with an acid of the anion in question if Compounds of formula (I) have a basic functionality. Agriculturally useful salts of Compounds of formula (I) encompass especially the acid addition salts of those acids whose cations and anions, respectively, have no adverse effect on the mode of action of Compounds of formula (1). Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting Compounds of formula (I) with an acid of the corresponding anion, preferably of hydrochloric acid, hydrobromic acid, sulfuric acid, phosphoric acid or nitric acid.

The term “N-oxide” includes Compounds of formula (I) which have at least one tertiary nitrogen atom that is oxidized to an N-oxide moiety. Of course, N-oxides can only be formed, if a nitrogen atom is present within Compounds of formula (I).

The organic moieties mentioned in the above definitions of the variables are—like the term halogen—collective terms for individual listings of the individual group members. The prefix Cₙ-Cₘ indicates in each case the possible number of carbon atoms in the group. The term “halogen” denotes in each case fluorine (F), bromine (Br), chlorine (Cl) or iodine (1), in particular fluorine, chlorine or bromine. The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl. The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like. The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms, e.g. 1 or 2 carbon atoms.

Examples of an alkoxy group are methoxy, ethoxy, n-propoxy, iso-propoxy, n-butyloxy, 2-butyloxy, iso-butyloxy, tert.-butyloxy, and the like. The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂—OC₂H₅, 2-(methoxy)ethyl, and 2-(ethoxy)ethyl. The term “alkylthio “(alkylsulfanyl: alkyl-S—)” as used herein refers to a straight-chain or branched saturated alkyl group having 1 to 10 carbon atoms, preferably 1 to 4 carbon atoms (=C₁-C₄-alkylthio), more preferably 1 to 3 carbon atoms, which is attached via a sulfur atom. The term “haloalkylthio” as used herein refers to an alkylthio group as mentioned above wherein the hydrogen atoms are partially or fully substituted by fluorine, chlorine, bromine and/or iodine. The term “alkenyl” as used herein denotes in each case a singly unsaturated hydrocarbon radical having usually 2 to 10, frequently 2 to 6, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like. The term “alkenyloxy” as used herein denotes in each case an alkenyl group as defined above, which is bonded via an oxygen atom and has usually from 2 to 10, preferably from 2 to 6 or from 2 to 4 carbon atoms. The term “alkynyl” as used herein denotes in each case a singly unsaturated hydrocarbon radical having usually 2 to 10, frequently 2 to 6, preferably 2 to 4 carbon atoms, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like. The term “alkynyloxy” as used herein denotes in each case an alkenyl group as defined above, which is bonded via an oxygen atom and has usually from 2 to 10, preferably from 2 to 6 or from 2 to 4 carbon atoms. The term “cycloalkylalkyl” refers to a cycloalkyl group as defined above which is bonded via an alkyl group, such as a C₁-C₆-alkyl group or a C₁-C₄-alkyl group, in particular a methyl group (=cycloalkylmethyl), to the remainder of the molecule. The term “cycloalkyl” as used herein and in the cycloalkyl moieties of cycloalkoxy and cycloalkylthio denotes in each case a monocyclic cycloaliphatic radical having usually from 3 to 10 or from 3 to 6 carbon atoms, such as cyclopropyl, cyclobutyl, cyclopentyl, cyclohexyl, cycloheptyl, cyclooctyl, cyclononyl and cyclodecyl or cyclopropyl, cyclobutyl, cyclopentyl and cyclohexyl. The term “cycloalkenyl” as used herein and in the cycloalkenyl moieties of cycloalkenyloxy and cycloalkenylthio denotes in each case a monocyclic singly unsaturated non-aromatic radical having usually from 3 to 10, e.g. 3, or 4 or from 5 to 10 carbon atoms, preferably from 3- to 8 carbon atoms. Exemplary cycloalkenyl groups include cyclopropenyl, cycloheptenyl or cyclooctenyl. The term “cycloalkenylalkyl” refers to a cycloalkenyl group as defined above which is bonded via an alkyl group, such as a C₁-C₆-alkyl group or a C₁-C₄-alkyl group, in particular a methyl group (=cycloalkenylmethyl), to the remainder of the molecule. The term “carbocycle” or “carbocyclyl” includes in general a 3- to 12-membered, preferably a 3- to 8-membered or a 5- to 8-membered, more preferably a 5- or 6-membered mono-cyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above. The term “heterocycle” or “heterocyclyl” includes in general 3- to 12-membered, preferably 3- to 8-membered or 5- to 8-membered, more preferably 5- or 6-membered, in particular 6-membered monocyclic heterocyclic non-aromatic radicals. The heterocyclic non-aromatic radicals usually comprise 1, 2, 3, 4, or 5, preferably 1, 2 or 3 heteroatoms selected from N, O and S as ring members, where S-atoms as ring members may be present as S, SO or SO₂. Examples of 5- or 6-membered heterocyclic radicals comprise saturated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piper-idinyl, piperazinyl, pyranyl, dihydropyranyl, tetrahydropyranyl, 1,3- and 1,4-dioxanyl, thiopyranyl, S.oxothiopyranyl, S-dioxothiopyranyl, dihydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-onyl and the like. The term “aryl” includes mono-, bi- or tricyclic aromatic radicals having usually from 6 to 14, preferably 6, 10, or 14 carbon atoms.

Exemplary aryl groups include phenyl, naphthyl and anthracenyl. Phenyl is preferred as aryl group. The term “hetaryl” includes monocyclic 5- or 6-membered heteroaromatic radicals comprising as ring members 1, 2, 3, or 4 heteroatoms selected from N, O and S. Examples of 5- or 6-membered heteroaromatic radicals include pyridyl, i.e. 2-, 3-, or 4-pyridyl, pyrimidinyl, i.e. 2-, 4-, or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3-, or 5-oxazolyl, isoxazolyl, i.e. 3-, 4-, or 5-isoxazolyl, thiazolyl, i.e. 2-, 3- or 5-thiazolyl, isothiazolyl, i.e. 3-, 4-, or 5-isothiazolyl, pyrazolyl, i.e. 1-, 3-, 4-, or 5-pyrazolyl, i.e. 1-, 2-, 4-, or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl. The term “hetaryl” also includes bicyclic 8 to 10-membered heteroaromatic radicals comprising as ring members 1, 2 or 3 heteroatoms selected from N, O and S, wherein a 5- or 6-membered heteroaromatic ring is fused to a phenyl ring or to a 5- or 6-membered heteroaromatic radical. Examples of a 5- or 6-membered heteroaromatic ring fused to a phenyl ring or to a 5- or 6-membered heteroaromatic radical include benzofuranyl, benzothienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like. These fused hetaryl radicals may be bonded to the remainder of the molecule via any ring atom of 5- or 6-membered heteroaromatic ring or via a carbon atom of the fused phenyl moiety. The terms ““benzyloxy” and “phenoxy” refer to a benzyl and a phenyl group, respectively, which are bonded via an oxygen atom to the remainder of the molecule. The terms “heterocyclylalkyl” and “hetarylalkyl” refer to heterocyclyl or hetaryl, respectively, as defined above which are bonded via a C₁-C₆-alkyl group or a C₁-C₄-alkyl group, in particular a methyl group (=heterocyclylmethyl or hetarylmethyl, respectively), to the remainder of the molecule. The term “arylalkyl” refers to aryl as defined above, which is bonded via C₁-C₆-alkyl group or a C₁-C₄-alkyl group, in particular a methyl group (=arylmethyl or phenylmethyl), to the remainder of the molecule, examples including benzyl, 1-phenylethyl, 2-phenylethyl, etc. The term “cyclic moiety” can refer to any cyclic groups, which are present in the compounds of the present invention, and which are defined above, e.g. cycloalkyl, cycloalkenyl, carbocycle, heterocycloalkyl, heterocycloalkenyl, heterocycle, aryl, hetaryl and the like.

The compositions may comprise auxiliaries. Suitable auxiliaries are solvents, carriers (e.g. liquid carriers, and solid carriers) or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders. Suitable solvents and liquid carriers are water and organic solvents, such as mineral oil fractions of medium to high boiling point, e.g. kerosene, diesel oil; oils of vegetable or animal origin; aliphatic, cyclic and aromatic hydro-carbons, e.g. toluene, paraffin, tetrahydronaphthalene, alkylated naphthalenes; alcohols, e.g. ethanol, propanol, butanol, benzyl alcohol, cyclohexanol; glycols; DMSO; ketones, e.g. cyclo-hexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof. Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof. Suitable surfactants are surface-active compounds, such as anionic, cationic, non-ionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulphates, phosphates, carboxylates, and mixtures thereof. Examples of sulfonates are alkylaryl-sulfonates, diphenylsulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulphates are sulphates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable non-ionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substititued fatty acid amides are fatty acid glucamides or fatty acid alkanolamides.

Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinyl alcohols, or vinyl acetate. Suitable cationic surfactants are quaternary surfactants, for example quaternary ammonium compounds with one or two hydrophobic groups, or salts of long-chain primary amines. Suitable amphoteric surfactants are alkylbetains and imidazolines.

Suitable block polymers are block polymers of the A-B or A-B-A type comprising blocks of polyethylene oxide and polypropylene oxide, or of the A-B—C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines. Suitable adjuvants are compounds, which have a neglectable or even no pesticidal activity themselves, and which improve the biological performance of the compound of formula (I) on the target. Examples are surfactants, mineral or vegetable oils, and other auxiliaries. Further examples are listed by Knowles, Adjuvants and additives, Agrow Reports DS256, T&F Informa UK, 2006, chapter 5. Suitable thickeners are polysaccharides (e.g. xanthan gum, carboxymethylcellulose), inorganic clays (organically modified or unmodified), polycarboxylates, and silicates. Suitable bactericides are bronopol and isothiazolinone derivatives such as alkylisothiazolinones and benzisothiazolinones. Suitable anti-freezing agents are ethylene glycol, propylene glycol, urea and glycerin. Suitable anti-foaming agents are silicones, long chain alcohols, and salts of fatty acids. Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants). Suitable tackifiers or binders are polyvinylpyrrolidones, polyvinylacetates, polyvinyl alcohols, polyacrylates, biological or synthetic waxes, and cellulose ethers.

The composition may comprise at least one fertilizer. The term “fertilizers” is to be understood as chemical compounds applied to promote plant and fruit growth. Fertilizers are typically applied either through the soil (for uptake by plant roots), through soil substituents (also for uptake by plant roots), or by foliar feeding (for uptake through leaves). The term also includes mixtures of one or more different types of fertilizers as mentioned below. The term “fertilizers” can be subdivided into several categories including: a) organic fertilizers (composed of decayed plant/animal matter), b) inorganic fertilizers (composed of chemicals and minerals) and c) urea-containing fertilizers.

Organic fertilizers include manure, e.g. liquid manure, semi-liquid manure, biogas manure, stable manure or straw manure, slurry, worm castings, peat, seaweed, compost, sewage, and guano. Green manure crops are also regularly grown to add nutrients (especially nitrogen) to the soil. Manufactured organic fertilizers include compost, blood meal, bone meal and seaweed extracts. Further examples are enzyme digested proteins, fish meal, and feather meal. The de-composing crop residue from prior years is another source of fertility. In addition, naturally occurring minerals such as mine rock phosphate, sulfate of potash and limestone are also considered inorganic fertilizers.

Inorganic fertilizers are usually manufactured through chemical processes (such as the Haber process), also using naturally occurring deposits, while chemically altering them (e.g. concentrated triple superphosphate). Naturally occurring inorganic fertilizers include Chilean sodium nitrate, mine rock phosphate, limestone, and raw potash fertilizers. The inorganic fertilizer may, in a specific embodiment, be a NPK fertilizer. “NPK fertilizers” are inorganic fertilizers formulated in appropriate concentrations and combinations comprising the three main nutrients nitrogen (N), phosphorus (P) and potassium (K) as well as typically S, Mg, Ca and trace elements. Other inorganic fertilizers include ammonium nitrate, calcium ammonium nitrate, ammonium sulfate nitrate, ammonium sulfate or ammonium phosphate.

Urea-containing fertilizer may, in specific embodiments, be urea, formaldehyde urea, urea ammonium nitrate (UAN) solution, urea sulfur, stabilized urea, urea based NPK-fertilizers, or urea ammonium sulfate. In case urea-containing fertilizers or urea are used or provided, it is particularly preferred that urease inhibitors as defined herein above may be added or additionally be present or be used at the same time or in connection with the urea-containing fertilizers. Urea-containing fertilizers are hydrolyzed by microorganisms, thereby releasing ammonia that in turn forms ammonium-ions. Urea-containing fertilizers may thus be considered as a storage form of ammonium.

Preferably, the fertilizer may be a solid or liquid ammonium-containing inorganic fertilizer such as an NPK fertilizer (which provides nitrogen, phosphorus, and potassium), ammonium nitrate, calcium ammonium nitrate, ammonium sulfate nitrate, ammonium sulfate or ammonium phosphate; a solid or liquid organic fertilizer such as liquid manure, semi-liquid manure, stable manure, biogas manure and straw manure, worm castings, compost, seaweed or guano, or an urea-containing fertilizer such as urea, formaldehyde urea, urea ammonium nitrate (UAN) solution, urea sulfur, stabilized urea, urea based NPK-fertilizers, urea ammonium sulfate, or a mixture thereof. More preferably, the fertilizer contains NH₄⁺-ions; more preferably the fertilizer is selected from solid or liquid ammonium-containing inorganic fertilizers.

Fertilizers may be provided in any suitable form, e.g. as solid coated or uncoated granules, in liquid or semi-liquid form, as sprayable fertilizer, or via fertigation etc.

Coated fertilizers may be provided with a wide range of materials. Coatings may, for example, be applied to granular or prilled nitrogen (N) fertilizer or to multi-nutrient fertilizers. Typically, urea is used as base material for most coated fertilizers. Alternatively, ammonium or NPK fertilizers are used is base material for coated fertilizers. The present invention, however, also envisages the use of other base materials for coated fertilizers, any one of the fertilizer materials defined herein. In certain embodiments, elemental sulfur may be used as fertilizer coating. The coating may be performed by spraying molten S over solid urea granules, followed by an application of sealant wax to close fissures in the coating. In a further embodiment, the S layer may be covered with a layer of organic polymers, preferably a thin layer of organic polymers. Further envisaged coated fertilizers may be provided by reacting resin-based polymers on the surface of the fertilizer granule. A further example of providing coated fertilizers includes the use of low permeability polyethylene polymers in combination with high permeability coatings. In specific embodiments the composition and/or thickness of the fertilizer coating may be adjusted to control, for example, the nutrient release rate for specific applications. The duration of nutrient release from specific fertilizers may vary, e.g. from several weeks to many months. The presence of nitrification inhibitors in a mixture with coated fertilizers may accordingly be adapted. It is, in particular, envisaged that the nutrient release involves or is accompanied by the release of a nitrification inhibitor according to the present invention. Coated fertilizers may be provided as controlled release fertilizers (CRFs). In specific embodiments these controlled release fertilizers are fully coated urea or N—P—K fertilizers, which are homogeneous, and which typically show a pre-defined longevity of release. In further embodiments, the CRFs may be provided as blended controlled release fertilizer products which may contain coated, uncoated and/or slow release components. In certain embodiments, these coated fertilizers may additionally comprise micronutrients. In specific embodiments these fertilizers may show a pre-defined longevity, e.g. in case of N—P—K fertilizers. Additionally envisaged examples of CRFs include patterned release fertilizers. These fertilizers typically show a pre-defined release patterns (e.g. hi/standard/lo) and a pre-defined longevity. In exemplary embodiments fully coated N—P—K, Mg and micronutrients may be delivered in a patterned release manner. Also envisaged are double coating approaches or coated fertilizers based on a programmed release. In further embodiments the fertilizer mixture may be provided as, or may comprise or contain a slow release fertilizer. The fertilizer may, for example, be released over any suitable period of time, e.g. over a period of 1 to 5 months, preferably up to 3 months. Typical examples of ingredients of slow release fertilizers are IBDU (isobutylidenediurea), e.g. containing about 31-32% nitrogen, of which 90% is water insoluble; or UF, i.e. an urea-formaldehyde product which contains about 38% nitrogen of which about 70% may be provided as water insoluble nitrogen; or CDU (crotonylidene diurea) containing about 32% nitrogen; or MU (methylene urea) containing about 38 to 40% nitrogen, of which 25-60% is typically cold water insoluble nitrogen; or MDU (methylene diurea) containing about 40% nitrogen, of which less than 25% is cold water insoluble nitrogen; or MO (methylol urea) containing about 30% nitrogen, which may typically be used in solutions; or DMTU (diimethylene triurea) containing about 40% nitrogen, of which less than 25% is cold water insoluble nitrogen; or TMTU (tri methylene tetraurea), which may be provided as component of UF products; or TMPU (tri methylene pentaurea), which may also be provided as component of UF products; or UT (urea triazone solution) which typically contains about 28% nitrogen. The fertilizer mixture may also be long-term nitrogen-bearing fertiliser containing a mixture of acetylene diurea and at least one other organic nitrogen-bearing fertiliser selected from methylene urea, isobutylidene diurea, crotonylidene diurea, substituted triazones, triuret or mixtures thereof.

The composition may comprise the fertilizer at a concentration of from 1 to 99.9 wt %, preferably of from 10 to 99 wt %, more preferably of from 10 to 98 wt %, most preferably of from 20 to 80 wt % based on the total weight of the composition. The composition may comprise at least 30 wt % of the fertilizer, preferably at least 50 wt %, more preferably at least 90 wt % of the fertilizer based on the total weigh of the composition. The composition typically comprises up to 99.9 wt % of the fertilizer based on the total weight of the composition, preferably up to 95 wt %, more preferably up to 90 wt %.

In case the fertilizer is an ammonium-containing fertilizer, the composition may comprise the ammonium-containing fertilizer in a concentration of at least 80 wt %, preferably at least 90 wt %, more preferably at least 95 wt % with regard to the total weight of the composition.

The composition may further comprise additional ingredients, for example at least one pesticidal compound. For example, the composition may additionally comprise at least one active substance selected from the group of fungicides, insecticides, nematicides, herbicides, safeners, micronutrients, biopesticides and/or growth regulators. In one embodiment, the pesticide is an insecticide. In another embodiment, the pesticide is a fungicide. In yet another embodiment the pesticide is a herbicide. The skilled worker is familiar with such pesticides, which can be found, for example, in the Pesticide Manual, 16th Ed. (2013), The British Crop Protection Council, London. Suitable insecticides are insecticides from the class of the carbamates, organo-phosphates, organochlorine insecticides, phenylpyrazoles, pyrethroids, neonicotinoids, spinosins, avermectins, milbemycins, juvenile hormone analogs, alkyl halides, organotin compounds nereistoxin analogs, benzoylureas, diacylhydrazines, METI acarizides, and insecticides such as chloropicrin, pymetrozin, flonicamid, clofentezin, hexythiazox, etoxazole, diafenthiuron, propargite, tetradifon, chlorofenapyr, DNOC, buprofezine, cyromazine, amitraz, hydramethylnon, acequinocyl, fluacrypyrim, rotenone, or their derivatives. Suitable fungicides are fungicides from the classes of dinitroanilines, allylamines, anilinopyrimidines, antibiotics, aromatic hydrocarbons, benzenesulfonamides, benzimidazoles, benzisothiazoles, benzophenones, benzothiadiazoles, benzotriazines, benzyl carbamates, carbamates, carboxamides, carboxylic acid diamides, chloronitriles cyanoacetamide oximes, cyanoimidazoles, cyclopropanecarboxamides, dicarboximides, dihydrodioxazines, dinitrophenyl crotonates, dithiocarbamates, dithiolanes, ethylphosphonates, ethylaminothiazolecarboxamides, guanidines, hydroxy-(2-amino)-pyrimidines, hydroxyanilides, imidazoles, imidazolinones, inorganic substances, isobenzofuranones, methoxyacrylates, methoxycarbamates, morpholines, N-phenylcarbamates, oxazolidinediones, oximinoacetates, oximinoacetamides, peptidylpyrimidine nucleosides, phenylacetamides, phenylamides, phenylpyrroles, phenylureas, phosphonates, phosphorothiolates, phthalamic acids, phthalimides, piperazines, piperidines, propionamides, pyridazinones, pyridines, pyridinylmethylbenzamides, pyrimidinamines, pyrimidines, pyrimidinonehydrazones, pyrroloquinolinones, quinazolinones, quinolines, quinones, sulfamides, sulfamoyltriazoles, thiazolecarboxamides, thiocarbamates, thiophanates, thiophenecarboxamides, toluamides, triphenyltin compounds, triazines, triazoles. Suitable herbicides are herbicides from the classes of the acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ether, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, ureas.

Suitable plant growth regulators are antiauxins, auxins, cytokinins, defoliants, ethylene modulators, ethylene releasers, gibberellins, growth inhibitors, morphactins, growth retardants, growth stimulators, and further unclassified plant growth regulators.

Suitable micronutrients are compounds comprising boron, zinc, iron, copper, manganese, chlorine, and molybdenum.

The composition may comprise one or more additional nitrification inhibitors. Examples of envisaged additional nitrification inhibitors are linoleic acid, alpha-linolenic acid, methyl p-coumarate, methyl ferulate, methyl 3-(4-hydroxyphenyl) propionate (MHPP), Karanjin, brachialacton, p-benzoquinone sorgoleone, 2-chloro-6-(trichloromethyl)-pyridine (nitrapyrin or N-serve), dicyandiamide (DCD, DIDIN), 3,4-dimethyl pyrazole phosphate (DMPP, ENTEC), 4-amino-1,2,4-triazole hydrochloride (ATC), 1-amido-2-thiourea (ASU), 2-amino-4-chloro-6-methylpyrimidine (AM), 2-mercapto-benzothiazole (MBT), 5-ethoxy-3-trichoromethyl-1,2,4-thiodiazole (terrazole, etridiazole), 2-sulfanilamidothiazole (ST), ammoniumthiosulfate (ATU), 3-methylpyrazol (3-MP), 3,5-dimethylpyrazole (DMP), 1,2,4-triazol thiourea (TU), N-(1H-pyrazolyl-methyl)acetamides such as N-((3(5)-methyl-1H-pyrazole-1-yl)methyl)acetamide, and N-(1H-pyrazolyl-methyl)formamides such as N-((3(5)-methyl-1H-pyrazole-1-yl)methyl formamide, N-(4-chloro-3(5)-methyl-pyrazole-1-ylmethyl)-formamide, N-(3(5),4-dimethyl-pyrazole-1-ylmethyl)-formamide, neem, products based on ingredients of neem, cyan amide, melamine, zeolite powder, catechol, benzoquinone, sodium terta board, zinc sulfate.

Suitable nitrification inhibitors also include
a) 2-(3,4-dimethyl-1H-pyrazol-1-yl)succinic acid (referred to as “DMPSA1” in the following) and/or 2-(4,5-dimethyl-1H-pyrazol-1-yl)succinic acid (referred to as “DMPSA2” in the following), and/or a derivative thereof, and/or a salt thereof;
b) glycolic acid addition salt of 3,4-dimethyl pyrazole (3,4-dimethyl pyrazolium glycolate, referred to as “DMPG” in the following), and/or an isomer thereof, and/or a derivative thereof;
c) citric acid addition salt of 3,4-dimethyl pyrazole (3,4-dimethyl pyrazolium citrate, referred to as “DMPC” in the following), and/or an isomer thereof, and/or a derivative thereof;
d) lactic acid addition salt of 3,4-dimethyl pyrazole (3,4-dimethyl pyrazolium lactate, referred to as “DMPL” in the following), and/or an isomer thereof, and/or a derivative thereof;
e) mandelic acid addition salt of 3,4-dimethyl pyrazole (3,4-dimethyl pyrazolium mandelate, referred to as “DMPM” in the following), and/or an isomer thereof, and/or a derivative thereof;
f) 1,2,4-triazole (referred to as “TZ” in the following), and/or a derivative thereof, and/or a salt thereof;
g) 4-Chloro-3-methylpyrazole (referred to as “ClMP” in the following), and/or an isomer thereof, and/or a derivative thereof, and/or a salt thereof;
h) a reaction adduct of dicyandiamide, urea and formaldehyde, or a triazonyl-formaldehyde-dicyandiamide adduct;
i) 2-cyano-1-((4-oxo-1,3,5-triazinan-1-yl)methyl)guanidine,
j) 1-((2-cyanoguanidino)methyl)urea;
k) 2-cyano-1-((2-cyanoguanidino)methyl)guanidine;
l) 3,4-dimethyl pyrazole phosphate;
m) allylthiourea, and
n) chlorate salts.

The composition may comprise from 1 to 90 wt % of the additional nitrification inhibitor, preferably 1 to 60 wt %, more preferably 1 to 30 wt % based on the total weight of the composition. The composition usually comprises at least 0.5 wt % of the additional nitrification inhibitor, more preferably at least 1 wt %, most preferably at least 2 wt %, especially preferably at least 10 wt %, and particularly preferably at least 20 wt % based on the total weight of the composition. The composition may contain up to 80 wt %, preferably up to 70 wt %, more preferably up to 40 wt %, most preferably up to 10 wt % of the additional nitrification inhibitor based on the total weight of the composition.

The composition may comprise one or more urease inhibitors. Examples of envisaged urease inhibitors include N-(n-butyl) thiophosphoric acid triamide (NBPT, Agrotain), N-(n-propyl) thiophosphoric acid triamide (NPPT), 2-nitrophenyl phosphoric triamide (2-NPT), further NXPTs known to the skilled person, phenylphosphorodiamidate (PPD/PPDA), hydroquinone, ammonium thiosulfate, and mixtures of NBPT and NPPT (see e.g. U.S. Pat. No. 8,075,659). Such mixtures of NBPT and NPPT may comprise NBPT in amounts of from 40 to 95% wt.-% and preferably of 60 to 80% wt.-% based on the total amount of active substances. Such mixtures are marketed as LIMUS, which is a composition comprising about 16.9 wt.-% NBPT and about 5.6 wt.-% NPPT and about 77.5 wt.-% of other ingredients including solvents and adjuvants.

The composition may comprise one or more plant growth regulators. Examples of envisaged plant growth regulators are antiauxins, auxins, cytokinins, defoliants, ethylene modulators, ethylene releasers, gibberellins, growth inhibitors, morphactins, growth retardants, growth stimulators, and further unclassified plant growth regulators.

The composition can be converted into customary types of agrochemical compositions, e.g. suspensions, dusts, powders, pastes, granules, pressings, and mixtures thereof. Preferably, the composition is in the form of a powder or a granule. Examples for composition types are suspensions (e.g. SC, OD, FS), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6^{th} Ed. May 2008, CropLife International. The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005. For the production of the composition,

Compounds of formula (I) are usually adsorbed on ZIF-8, in particular when the composition is a powder or a granule. To this end, Compounds of formula (I), or a solution comprising Compounds of formula (I) may be contacted with ZIF-8, wherein ZIF-8 is typically in the form of a powder, or in the form of granules. In case a solution of Compounds of formula (I) is applied, the solvent is typically removed after the contacting by elevated temperatures or reduced pressure.

In case ZIF-8 is in form of a powder, the powder may subsequently be granulated. Accordingly, the invention typically relates to a composition in which Compounds of formula (I) are adsorbed on ZIF-8, i.e. the composition contains up to 99 wt %, preferably up to 95 wt %, more preferably up to 80 wt %, most preferably up to 70 wt %, especially preferably up to 50 wt %, and in particular up to 30 wt % of the weight of Compounds of formula (I) relative to the weight of Compounds of formula (I) that can be adsorbed at maximum by the amount of ZIF-8 present.

In the composition, essentially the whole amount of Compounds of formula (I) is adsorbed on the ZIF-8 material present, i.e. at least 50 mol %, preferably at least 80 mol %, more preferably at least 95 mol %, and in particular at least 99 mol % of Compounds of formula (I) are adsorbed on ZIF-8 at 25° C. and 1013 hPa gas phase pressure, wherein the gas phase consists of Compounds of formula (I), and wherein the system is in equilibrium. The adsorption of Compounds of formula (I) under these conditions can be measured by UV-, or IR-spectroscopy, e.g. NIR-spectroscopy.

Examples for composition types and their preparation are:

i) Water-dispersible granules and water-soluble granules (WG, SG) 50-80 wt % of the fertilizer composition is ground finely with addition of dispersants and wetting agents (e.g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the fertilizer composition.

ii) Water-dispersible powders and water-soluble powders (WP, SP, WS) 50-80 wt % of the fertilizer composition is ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the fertilizer composition.

iii) Dustable powders (DP, DS) 1-10 wt % of the fertilizer composition is ground finely and mixed intimately with solid carrier (e.g. finely divided kaolin) ad 100 wt %.

iv) Granules (GR, FG) 0.5-30 wt % of the fertilizer composition is ground finely and associated with solid carrier (e.g. silicate) ad 100 wt %. Granulation is achieved by extrusion, spray-drying or the fluidized bed.

The compositions types i) to iv) may optionally comprise further auxiliaries, such as 0.1-1 wt % bactericides, 5-15 wt % anti-freezing agents, 0.1-1 wt % anti-foaming agents, and 0.1-1 wt % colorants.

Powders for dry treatment (DS), water-dispersible powders for slurry treatment (WS), and water-soluble powders (SS) are usually employed for the purposes of treatment of plant propagation materials, particularly seeds. The compositions in question give, after two-to-tenfold dilution, concentrations of the Compounds of formula (I) of from 0.01 to 60% by weight, preferably from 0.1 to 40% by weight, in the ready-to-use preparations. Application can be carried out before or during sowing. Methods for applying the composition on to plant propagation material, especially seeds include dressing, coating, pelleting, dusting, soaking and in-furrow application methods of the propagation material. Preferably, the fertilizer composition is applied on to the plant propagation material by a method such that germination is not induced, e.g. by seed dressing, pelleting, coating and dusting.

The invention also relates to the use of ZIF-8 for reducing the evaporation rate of Compounds of formula (I). Typically, the use comprises the contacting, e.g. by mixing, of ZIF-8 with Compounds of formula (I), preferably in a way that leads to the adsorption of Compounds of formula (I) on ZIF-8, as described above. The term “reducing the evaporation rate” refers to a situation in which the evaporation rate of the composition is determined at 1013 hPa at a predetermined temperature and after a predetermined time-interval and compared to free Compounds of formula (I), which have not been contacted with ZIF-8.

Typically, the evaporation rate may be reduced by at least 50%, preferably at least 70%, and in particular at least 90% at 35° C. after a time interval of 23 hours. In one embodiment, the evaporation rate is reduced by at least 50%, preferably at least 70%, and in particular at least 90% at 60° C. after a time interval of 32 hours. In another embodiment, the evaporation rate is reduced by at least 50%, preferably at least 70%, and in particular at least 90% at 100° C. after a time interval of 4 hours. Besides the temperature and the time interval, the evaporation rate depends on the loading of Compounds of formula (I) on ZIF-8, i.e. the weight ratio of Compounds of formula (I) to ZIF-8. Typically, the evaporation rate is measured at a weight ratio of Compounds of formula (I) to ZIF-8 of 1:1.

The invention also relates to a method for producing the composition comprising the step a) of adsorbing Compounds of formula (I) on ZIF-8.

Step a) is typically carried out at a temperature of 10° C. to 50° C., preferably from 20° C. to 30° C. In one embodiment step a) may be carried out at a temperature of 50° C. to 150° C., preferably 50° C. to 120° C., preferably 60° C. to 110° C., more preferably from 70° C. to 100° C. Compounds of formula (I) are usually applied in liquid form. In case they are liquid themselves, Compounds of formula (I) may be applied in pure form or as solutions in water or organic liquids. In case Compounds of formula (I) are solids, they are usually applied as solutions in water or organic liquids. For adsorbing Compounds of formula (I) on ZIF-8, the liquid Compounds of formula (I), or a solution containing Compounds of formula (I), are typically contacted with ZIF-8 and optionally incubated at a temperature of 10° C. to 40° C. for a period of from 10 min to 300 min. In case Compounds of formula (I) are applied as a solution in a solvent, the solvent may be removed by evaporation at reduced pressure and/or elevated temperatures. The contacting as described above results in an adsorption of Compounds of formula (I) on the surface of ZIF-8, including the internal and external surface.

In one embodiment, the method for production of the composition comprises the steps of

a) adsorbing Compounds of formula (I) on ZIF-8; and
b) co-granulating Compounds of formula (I) adsorbed on ZIF-8 with a fertilizer;
wherein the composition is in the form of granules.

Typically, the temperature in step b) may be from 50° C. to 150° C., preferably 60 to 120° C., more preferably from 70 to 100° C. The temperature may be at least 80° C., preferably at least 90° C. The temperature is typically below 150° C., preferably below 130° C. The term “the temperature in step b” relates to a situation in which the temperature is defined for the whole length of step b), or only for a short period in the process step. Preferably, the temperature is defined for the whole length of step b).

Suitable co-granulation techniques are known to the skilled person. Typically, the co-granulation process is a wet granulation process, in which a spray liquid—typically containing water, ethanol, or isopropanol—is sprayed on the powder to be granulated. In one embodiment, the powder to be co-granulated comprises both the Compounds of formula (I) adsorbed on ZIF-8 and the fertilizer at the beginning of step b). In this case Compounds of formula (I) adsorbed on ZIF-8 are mixed with the fertilizer prior to step b). In another embodiment, Compounds of formula (I) adsorbed on ZIF-8 and the fertilizer are only mixed during the co-granulation process step b), i.e. that one component is initially charged to the granulation device, and the other component is gradually added to the device during the co-granulation process.

In case of a wet granulation process, a spray liquid may be sprayed on the powder before or during granulation, and may contain a binder, such as polyvinyl pyrrolidone. Typical co-granulation processes include fluidized bed granulation and spouted bed granulation. The spray liquid is typically removed after or during co-granulation by evaporation at elevated temperatures.

It has been surprisingly found that the composition displays a reduced evaporation rate as compared to Compounds of formula (I) alone, which effect is also and especially present at elevated temperatures that are typical for granulation processes. The evaporation rate of Compounds of formula (I) is especially high in case Compounds of formula (I) are in the form of a spray, which is particularly detrimental if they are applied in a wet granulation process in dissolved form in the spray liquid. Once Compounds of formula (I) are adsorbed on ZIF-8 in step a), however, they may be applied in granulation methods that are carried out at elevated temperatures, thereby preventing the evaporation and loss of Compounds of formula (I) during granulation.

Accordingly, in one embodiment the method for producing the composition comprises the steps of

a) adsorbing Compounds of formula (I) on ZIF-8 powder; and
b) granulating the obtained powder;
wherein the temperature in step b) is from 50° C. to 150° C.

In another embodiment the method for producing the composition comprises the steps of

a) adsorbing Compounds of formula (I) on ZIF-8 powder or granules; and
b) co-granulating the obtained powder or granules with fertilizer;
wherein step b) is carried out at a temperature of from 50° C. to 150° C.

In another embodiment the method for producing the composition comprises the steps of

a) adsorbing Compounds of formula (I) on ZIF-8 powder or granules; and
b) co-granulating the obtained powder or granules with fertilizer by a wet granulation process;
wherein step b) is carried out at a temperature of from 50° C. to 150° C.

The invention also relates to the use of the composition for producing granules comprising Compounds of formula (I) and a fertilizer, in particular for producing granules comprising Compounds of formula (I), ZIF-8, and a fertilizer. Typically, the granules are produced at a temperature of from 50° C. to 150° C., preferably 60 to 110° C., more preferably from 70 to 100° C. The temperature may be at least 80° C., preferably at least 90° C. The temperature is typically below 150° C., preferably below 130° C. The granules may comprise auxiliaries as defined above, which may be added in any step of the process for producing the granules.

The invention also relates to a method for reducing nitrification, comprising treating plant propagation material, such as seeds, a plant growing on soil or soil substituents and/or the locus or soil or soil substituents where the plant is growing or is intended to grow with the composition. The invention also relates to a method for fertilization, comprising treating plant propagation material, such as seeds, a plant growing on soil or soil substituents and/or the locus or soil or soil substituents where the plant is growing or is intended to grow with the composition. Another object is the use of the composition for fertilizing plant propagation material, a plant growing on soil or soil substituents and/or the locus or soil or soil substituents where the plant is growing or is intended to grow.

The term “reducing nitrification” or “reduction of nitrification” as used herein refers to a slowing down or stopping of nitrification processes, e.g. by retarding or eliminating the natural transformation of ammonium into nitrate. Such reduction may be a complete or partial elimination of nitrification at the plant or locus where composition is applied. For example, a partial elimination may result in a residual nitrification on or in the plant, or in or on the soil or soil substituents where a plant grows or is intended to grow of 90% to 1%, e.g. 90%, 85%, 80%, 70%, 60%, 50%, 40%, 30%, 20%, 10% or less than 10%, e.g. 5% or less than 5% in comparison to a control situation in which the composition is not used. In certain embodiments, a partial elimination may result in a residual nitrification on or in the plant or in or on the soil or soil substituents where a plant grows or is intended to grow of below 1%, e.g. at 0.5%, 0.1% or less in comparison to a control situation where the nitrification inhibitor is not used.

In one embodiment, the plant to be treated according to the method of the invention is an agricultural plant. “Agricultural plants” are plants of which a part (e.g. seeds) or all is harvested or cultivated on a commercial scale or which serve as an important source of feed, food, fibers (e.g. cotton, linen), combustibles (e.g. wood, bioethanol, biodiesel, biomass) or other chemical compounds. Preferred agricultural plants are for example cereals, e.g. wheat, rye, barley, triticale, oats, corn, sorghum or rice, beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, oil-seed rape, canola, linseed, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, canola, sugar cane or oil palm; tobacco; nuts; coffee; tea; bananas; vines (table grapes and grape juice grape vines); hop; turf; natural rubber plants.

In a further embodiment, the plant to be treated according to the method of the invention is a horticultural plant. The term “horticultural plants” are to be understood as plants which are commonly used in horticulture, e.g. the cultivation of ornamentals, vegetables and/or fruits. Examples for ornamentals are turf, geranium, pelargonia, petunia, begonia and fuchsia. Examples for vegetables are potatoes, tomatoes, peppers, cucurbits, cucumbers, melons, watermelons, garlic, onions, carrots, cabbage, beans, peas and lettuce and more preferably from tomatoes, onions, peas and lettuce. Examples for fruits are apples, pears, cherries, strawberry, citrus, peaches, apricots and blueberries.

In a further embodiment, the plant to be treated according to the method of the invention is an ornamental plant. “Ornamental plants” are plants which are commonly used in gardening, e.g. in parks, gardens and on balconies. Examples are turf, geranium, pelargonia, petunia, begonia and fuchsia.

In another embodiment of the present invention, the plant to be treated according to the method of the invention is a silvicultural plant. The term “silvicultural plant” is to be understood as trees, more specifically trees used in reforestation or industrial plantations. Industrial plantations generally serve for the commercial production of forest products, such as wood, pulp, paper, rubber tree, Christmas trees, or young trees for gardening purposes. Examples for silvicultural plants are conifers, like pines, in particular Pinus spec., fir and spruce, eucalyptus, tropical trees like teak, rubber tree, oil palm, willow (Salix), in particular Salix spec., poplar (cottonwood), in particular Populus spec., beech, in particular Fagus spec., birch, oil palm, and oak.

Suitable application methods include inter alia soil treatment, seed treatment, in furrow application, and foliar application. Soil treatment methods include drenching the soil, drip irrigation (drip application onto the soil), dipping roots, tubers or bulbs, or soil injection. Seed treatment techniques include seed dressing, seed coating, seed dusting, seed soaking, and seed pelleting. In furrow applications typically include the steps of making a furrow in cultivated land, seeding the furrow with seeds, applying the pesticidally active compound to the furrow, and closing the furrow.

The treatment according to the methods of application and uses according to the invention may also be carried out by fertigation. The term “fertigation” as used herein refers to the application of fertilizers, optionally soil amendments, and optionally other water-soluble products together with water through an irrigation system to a plant or to the locus where a plant is growing or is intended to grow, or to a soil substituent as defined herein below. For example, liquid fertilizers or dissolved fertilizers may be provided via fertigation directly to a plant or a locus where a plant is growing or is intended to grow. Likewise, the composition, or fertilizers, optionally in combination with additional nitrification inhibitors, may be provided via fertigation to plants or to a locus where a plant is growing or is intended to grow. The composition, the fertilizers, and optionally the additional nitrification inhibitors according to the present invention may be provided together, e.g. dissolved in the same charge or load of material (typically water) to be irrigated. In further embodiments, the composition, the fertilizers and optionally the additional nitrification inhibitors may be provided at different points in time. For example, the composition may be fertigated first, followed by the fertilizer and optionally the additional nitrification inhibitors, or preferably, the fertilizer and optionally the additional nitrification inhibitors may be fertigated first, followed by the composition. Also envisaged is a repeated fertigation of the composition and fertilizers and optionally nitrification inhibitors according to the present invention, either together or intermittently, e.g. every 2 hours, 6 hours, 12 hours, 24 hours, 2 days, 3 days, 4 days, 5 days, 6 days or more.

The term “plant propagation material” refers to all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “seed” embraces seeds and plant propagules of all kinds including but not limited to true seeds, seed pieces, suckers, corms, bulbs, fruit, tubers, grains, cuttings, cut shoots and the like, and means in a preferred embodiment true seeds.

The term “soil substituent” as used herein refers to a substrate which is able to allow the growth of a plant and does not comprise usual soil ingredients. This substrate is typically an anorganic substrate which may have the function of an inert medium. It may, in certain embodiments, also comprise organic elements or portions. Soil substituents may, for example, be used in hydroculture or hydroponic approaches, i.e. wherein plants are grown in soilless medium and/or aquatic based environments. Examples of suitable soil substituents, which may be used in the context of the present invention, are perlite, gravel, biochar, mineral wool, coconut husk, phyllosilicates, i.e. sheet silicate minerals, typically formed by parallel sheets of silicate tetrahedra with Si₂O₅ or a 2:5 ratio, or clay aggregates, in particular expanded clay aggregates with a diameter of about 10 to 40 mm. Particularly preferred is the employment of vermiculite, i.e. a phyllosilicate with 2 tetrahedral sheets for every one octahedral sheet present. The use of soil substituents may, in specific embodiments, be combined with fertigation or irrigation as defined herein.

The term “where the plant is intended to grow” refers to a place, which is chosen by an applicant, such as a farmer, for growing a desired plant, such as a crop plant.

For the methods of application, the application rates of Compounds of formula (I) are between 0.01 g and 5 kg of active ingredient per hectare, preferably between 1 g and 1 kg of active ingredient per hectare, especially preferred between 50 g and 300 g of active ingredient per hectare depending on different parameters such as the specific active ingredient applied and the plant species treated.

For the methods of application, the application rates of fertilizers may be between 10 kg and 1000 kg per hectare, preferably between 50 kg and 700 kg per hectare, in certain cases between 50 kg and 400 kg per hectare.

The term “treatment of seeds” comprises all suitable seed treatment techniques known in the art, such as seed dressing, seed coating, seed dusting, seed soaking, seed pelleting, and in-furrow application methods. Preferably, the seed treatment application of the active compound is carried out by spraying or by dusting the seeds before sowing of the plants and before emergence of the plants.

The invention also relates to plant propagation material comprising the composition. In treatment of plant propagation materials such as seeds, e.g. by dusting, coating or drenching seed, amounts of the composition of from 0.1 to 1000 g, preferably from 1 to 1000 g, more preferably from 1 to 100 g and most preferably from 5 to 100 g, per 100 kilogram of plant propagation material are generally required.

Preferred plant propagation materials are seeds coated with or containing the composition. The term “coated with and/or containing” generally signifies that the fertilizer composition is for the most part on the surface of the propagation product at the time of application, although a greater or lesser part of the ingredients may penetrate into the propagation product, depending on the method of application. When the said propagation product is (re)planted, it may absorb the active ingredient.

Suitable seed is for example seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment of seeds from plants, which have been modified by mutagenisis or genetic engineering, and which e.g. tolerate the action of herbicides or fungicides or insecticides.

Conventional seed treatment formulations include for example powders for dry treatment DS, water dispersible powders for slurry treatment WS, and water-soluble powders SS. These formulations can be applied to the seed diluted or undiluted. Application to the seeds is carried out before sowing, either directly on the seeds or after having pregerminated the latter. Preferably, the formulations are applied such that germination is not included.

In the treatment of seed, the application rates of the sum of ZIF-8 and the compounds of formula (I) are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed, e.g. from 1 g to 100 g or from 5 g to 100 g per 100 kg of seed.

The invention therefore also relates to seed comprising the composition. The amount of the sum of ZIF-8 and the compounds of formula (I) will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

In one embodiment, the methods of application include the treatment of the plant propagation material, the plant and/or the locus or soil or soil substituents where the plant is growing or is intended to grow, with an additional compound selected from a fertilizer, nitrification inhibitor, urease inhibitor, plant growth regulator, and pesticide.

The application of the composition and the additional compound may be carried out simultaneously or with a time lag, wherein either said additional compound or the composition may be applied first. Preferably, said time lag is an interval of 1 day, 2 days, 3 days, 4 days, 5 days, 6 days, 1 week, 2 weeks or 3 weeks. In case of application with a time lag, the composition may be applied first and then the additional compound. In another embodiment, in a first step the composition is applied to plant propagation material, to a plant and/or to the locus where the plant is growing or is intended to grow and in a second step the additional compound is applied to the plant propagation material, the plant and/or to the locus where the plant is growing or is intended to grow, wherein the application of the composition in the first step and the additional compound in the second step is carried out with a time lag of at least 1 day, 2 days, 3 days, 4 days, 5, days, 6 days, 1 week, 2 weeks or 3 weeks.

In other embodiments of application with a time lag, the additional compound may be applied first and then the composition may be applied. In a further preferred embodiment of the method, in a first step the additional compound is applied to a plant and/or to the locus where the plant is growing or is intended to grow and in a second step the composition is applied to plant propagation material, to a plant and/or to the locus where the plant is growing or is intended to grow, wherein the application of a the additional compound in the first step and the composition in the second step is carried out with a time lag of at least 1 day, 2 days, 3 days, 4 days, 5, days, 6 days, 1 week, 2 weeks or 3 weeks.

The methods of application and uses of the composition may be a single application or use, or it may be a repeated application or use. As single application or use, the composition may be provided to their target sites, e.g. soil or loci, or objects, e.g. plants, only once in a physiologically relevant time interval, e.g. once a year, or once every 2 to 5 years, or once during the lifetime of a plant.

In other embodiments, the application or use may be repeated at least once per time period, e.g. the composition may be used for reducing nitrification at their target sites or objects two times within a time interval of days, weeks or months. The term “at least once” as used in the context of a use of the composition means that the composition may be used two times, or several times, i.e. that a repetition or multiple repetitions of an application or treatment with the composition may be envisaged. Such a repetition may be a 2 times, 3 times, 4 times, 5 times, 6 times, 7 times, 8 times, 9 times, 10 times or more frequent repetition of the use.

The invention also relates to a method for treating a fertilizer, comprising contacting the composition with the fertilizer. The contacting may be achieved by mixing, co-grinding, co-granulating, co-precipitation, adsorption and other techniques familiar to the skilled person and as described above.

The invention also relates to a kit-of-parts comprising a ZIF-8 and a Compound of formula (I) as individual components of the composition or partially premixed components, e.g. components comprising ZIF-8 and a Compound of formula (I) and/or mixing partners (e.g. further nitrification inhibitors, urease inhibitors, pesticides, plant growth regulators, micronutrients, auxiliaries etc.) as defined above, which may be mixed by the user himself, e.g. in a spray tank, and further auxiliaries may be added, if appropriate.

The following examples illustrate the invention.

### EXAMPLES

ZIF-8 was purchased as Basolite® Z1200 from SigmaAldrich.

Zeolite Beta was purchased from Alfa Aesar.

Compound A: 1-chloro-4-(prop-2-ynoxymethyl)benzene (corresponding to compound 1-15 in Table A).

### Working Example 1: Compound a on ZIF-8

ZIF-8 powder (10 g) was placed in a porcelain dish. The ZIF-8 powder was in the activated stage with virtually nothing adsorbed in the pores of the material. Compound A (3 g, below 2% impurities) was slowly added at 20 to 25° C. to the manually agitated ZIF-8 powder over a period of 15 minutes to result in Sample A containing 23 wt % of Compound A based on the total weight of Sample A.

### Working Example 2: Compound a on ZIF-8 at Different Loadings

ZIF-8 was loaded with Compound A as described in Working Example 1 with different amounts of Compound A, thereby resulting in the Samples B-H of Table 1.

TABLE 1
Loading of ZIF-8 with different amounts of Compound A in [wt %]

| | Sample B | Sample C | Sample D | Sample E | Sample F |
|---|---|---|---|---|---|
| Loading [wt %] relative | 16.6 | 28.6 | 37.5 | 44.4 | 50 |
| to the total weight of | | | | | |
| the sample | | | | | |

| | Sample G | Sample H |
|---|---|---|
| Loading [wt %] relative | 54.5 | 58.3 |
| to the total weight of | | |
| the sample | | |

### Comparative Working Example 1: Compound a on Zeolite Beta

Zeolite Beta powder (10 g) were placed in a porcelain dish. The zeolite Beta powder was in the activated stage with virtually nothing adsorbed in the pores of the material. Compound A (3 g, below 2% impurities) was slowly added at 20 to 25° C. to the manually agitated zeolite Beta powder over a period of 15 minutes to result in Sample K containing 23 wt % of Compound A based on the total weight of Sample K.

### Example-1: Volatility Measurement at 35° C.

Five beakers B1-B5 were prepared containing samples as specified in Table 2

TABLE 2
Samples in Beakers 1-5.

| B 1 | B 2 | B 3 | B 4 | B 5 |
|---|---|---|---|---|
| Compound A | ZIF-8 | Sample A (8.7 g | Zeolite | Sample K (8.7 g |
| (2 g) | (6.7 g) | containing 2 g | Beta | containing 2 g |
| | | of Compound A) | (6.7 g) | of Compound A) |

All 5 beakers were mounted in an oil bath such that half of the beakers were immersed. The temperature of the oil bath was set to 35° C. During the heating process, the beakers were periodically taken out of the oil bath, dried, and weighted. The total mass of the beaker with samples was noted and compared to the initial weight of the beakers with samples. The weight loss attributed to Compound A was calculated from the total weight loss of the sample by taking the loading of the respective sample with Compound A into account. A comparison of the weight loss of Compound A, and the whole sample weight loss in the all 5 beakers after 23 hours at 35° C. was shown in the Table 3.

TABLE 3
Weight loss of samples in [%] relative to the initial weight.

| Sample | B1 | B2 | B3 | B4 | B5 |
|---|---|---|---|---|---|
| Weight loss of | 4.22 | — | 0.27 | — | 26.48 |
| Compound A | | | | | |
| Total sample | 4.22 | 0.14 | 0.05 | 1.10 | 6.07 |
| weight loss | | | | | |

### Example-2: Volatility Measurement at 60° C.

Five beakers B1-B5 were prepared and treated as described in Example-1 with the differences that the oil bath was set to 60° C. and that the weight loss was recorded after 32 hours. Table 4 showed a comparison of the weight loss Compound A, and the whole sample weight loss in the all 5 beakers after 32 hours at 60° C.

TABLE 4
Weight loss of samples in [%] relative to the initial weight.

| Sample | B1 | B2 | B3 | B4 | B5 |
|---|---|---|---|---|---|
| Weight loss of | 37.97 | — | 0.83 | — | 41.78 |
| Compound A | | | | | |
| Total sample | 37.97 | 0.10 | 0.19 | 5.69 | 9.58 |
| weight loss | | | | | |

### Example-3: Volatility Measurement at 100° C.

Five beakers B1-B5 were prepared and treated as described in Example-1 with the differences that the oil bath was set to 100° C. and that the weight loss was recorded after hours. Table 5 showed a comparison of the weight loss Compound A, and the whole sample weight loss in the all 5 beakers after hours at 100° C.

TABLE 5
Weight loss of samples in [%] relative to the initial weight.

| Sample | B1 | B2 | B3 | B4 | B5 |
|---|---|---|---|---|---|
| Weight loss of | 68.82 | — | 1.87 | — | 44.69 |
| Compound A | | | | | |
| Total sample | 68.82 | 0.49 | 0.44 | 8.64 | 10.20 |
| weight loss | | | | | |

### Example-4: Volatility Measurement at 60° C. with Different Loading for 24 or 72 Hours

Five beakers B6-B10 were prepared containing samples as specified in Table 6.

TABLE 6
Samples in Beakers 6-10.

| B6 | B7 | B8 | B9 | B10 |
|---|---|---|---|---|
| Sample B (6 g | Sample C (7 g | Sample D (8 g | Sample E (9 g | Sample F (10 g |
| containing 1 g | containing 2 g | containing 3 g | containing 4 g | containing 5 g |
| of Compound A) | of Compound A) | of Compound A) | of Compound A) | of Compound A) |

All 5 beakers B6-B10 were mounted in an oil bath such that half of the beakers were immersed. The temperature of the oil bath was set to 60′C. During the heating process, the beakers were periodically taken out of the oil bath, dried, and weighted. The total mass of the beaker with samples was noted and compared to the initial weight of the beakers with samples. The weight loss attributed to Compound A was calculated from the total weight loss of the sample by taking the loading of the respective sample with Compound A into account. A comparison of the weight loss of Compound A, and the whole sample weight loss in the all 5 beakers after 24 and 72 hours at 60° C. was shown in the Table 7.

TABLE 7
Weight loss of samples in [%] relative to
the initial weight after 24 and 72 hours.

| Sample | B6 | B7 | B8 | B9 | B10 |
|---|---|---|---|---|---|
| Weight loss of | 1.38 | −0.11 | 7.59 | 6.76 | 6.99 |
| Compound A | | | | | |
| after 24 hours | | | | | |
| Total sample | 0.23 | −0.03 | 2.86 | 3.01 | 3.49 |
| weight loss | | | | | |
| after 24 hours | | | | | |
| Weight loss of | 1.17 | 0.46 | 15.21 | 16.62 | 18.1 |
| Compound A | | | | | |
| after 72 hours | | | | | |
| Total sample | 0.19 | 0.13 | 5.72 | 7.40 | 9.05 |
| weight loss | | | | | |
| after 72 hours | | | | | |

### Example-5: Volatility Measurement at 60° C. with Different Loading for 24 or 72 Hours

Five beakers B11-B15 were prepared containing samples as specified in Table 8.

TABLE 8
Samples in Beakers 11-15.

| B11 | B12 | B13 | B14 | B15 |
|---|---|---|---|---|
| Sample E (9 g | Sample F (10 g | Sample G (11 g | Sample H (12 g | Compound A |
| containing 4 g | containing 5 g | containing 6 g | containing 7 g | (7 g) |
| of Compound A) | of Compound A) | of Compound A) | of Compound A) | |

All 5 beakers B11-B15 were mounted in an oil bath such that half of the beakers were immersed. The temperature of the oil bath was set to 60′C. During the heating process, the beakers were periodically taken out of the oil bath, dried, and weighted. The total mass of the beaker with samples was noted and compared to the initial weight of the beakers with samples. The weight loss attributed to Compound A was calculated from the total weight loss of the sample by taking the loading of the respective sample with Compound A into account. A comparison of the weight loss of Compound A, and the whole sample weight loss in the all 5 beakers after 24 and 72 hours at 60° C. was shown in the Table 9.

TABLE 9
Weight loss of samples in [%] relative to
the initial weight after 24 and 72 hours.

| Sample | B11 | B12 | B13 | B14 | B15 |
|---|---|---|---|---|---|
| Weight loss of | 6.66 | 7.06 | 5.80 | 4.92 | 9.02 |
| Compound A | | | | | |
| after 24 hours | | | | | |
| Total sample | 2.96 | 3.53 | 3.17 | 2.87 | 9.02 |
| weight loss | | | | | |
| after 24 hours | | | | | |
| Weight loss of | 16.64 | 18.01 | 16.64 | 14.47 | 26.99 |
| Compound A | | | | | |
| after 72 hours | | | | | |
| Total sample | 7.40 | 9.01 | 9.08 | 8.44 | 26.99 |
| weight loss | | | | | |
| after 72 hours | | | | | |

### Example 6: Nitrogen Retention in Field Trials

An area of sandy soil, previously untreated with fertilizer or other agricultural products near the field station Limburgerh of, was treated as follows. Drainage pipes of 20 cm in length and 12 cm diameter were inserted 10 cm deep into the soil. A treatment as indicated in a line of Table 10 was added on top of the soil under which a pipe was buried. At the beginning of the experiment and after three weeks, the pipes were dug out of the soil with its content, the content emptied into a bag and frozen on site. The soil was then analyzed for NH₄-nitrogen content in the laboratory according to the following method: Samples were defrosted for 24 h, then sieved through a 5-6 mm mesh sieve. 200 g of the thus homogenized samples were put into 1 L plastic bottles and 600 mL of a K₂S₄ solution (1% (w/w) K₂SO₄ in water) were added. The samples were then shaken for 2 h end over end. The solution was filtered to remove the soil and 50 mL of the filtrate retained for analysis in a Continuous Flow Analyzer for NH₄-nitrogen content.

TABLE 10
Field trial results

| | Incorpora- | | NH₄- |
|---|---|---|---|
| | tion Depth | | nitrogen |
| Treatment | (cm) | Timepoint | (kg/ha) |
| none | — | 3 weeks after application | 0 |
| Ammonium Sulfate | 0 | At time of application | 75 |
| Ammonium Sulfate | 0 | 3 weeks after application | 8 |
| Ammonium Sulfate + | 0 | 3 weeks after application | 50 |
| Sample A | | | |
| Ammonium sulfate + | 0 | 3 weeks after application | 8 |
| Compound A | | | |
| Ammonium Sulfate + | 0 | 3 weeks after application | 30 |
| MOF | | | |

1. A composition comprising
a) zeolitic imidazolate framework ZIF-8; and
b) a compound of formula (I) 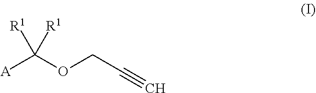
or a stereoisomer, salt, tautomer or N-oxide thereof, wherein the variables have the following meaning:
R¹, R² are independently H;
C₁-C₆-alkyl, C₂-C₆-alkenyl, C₂-C₆-alkynyl, C₁-C₆-haloalkyl, C₁-C₄-alkoxy-C₁-C₄-alkyl C₁-C₆-alkoxy, C₂-C₆-alkenyloxy, C₂-C₆-alkynyloxy, which groups are unsubstituted or substituted by one or more, same or different R^{e}; or
C₃-C₈-cycloalkyl, C₃-C₈-cycloalkenyl, heterocyclyl, aryl, hetaryl, C₃-C₈-cycloalkyl-C₁-C₆-alkyl, C₃-C₈-cycloalkenyl-C₁-C₆-alkyl, heterocyclyl-C₁-C₆-alkyl, aryl-C₁-C₆-alkyl, hetaryl-C₁-C₆-alkyl, phenoxy, or benzyloxy, wherein the cyclic moieties are unsubstituted or substituted by one or more, same or different R^{a};
A phenyl, which is unsubstituted or substituted with one or more, same or different R^{A};
R^{A} CN, halogen, NO₂, OR^{b}, NR^{c}R^{d}, C(Y)R^{b}, C(Y)OR^{b}, C(Y)NR^{c}R^{d}, S(Y)ₘR^{b}, S(Y)ₘOR^{b};
C₁-C₆-alkyl, C₂-C₆-alkenyl, C₂-C₆-alkynyl, C₁-C₆-haloalkyl, C₁-C₆-alkoxy, C₁-C₆-alkylthio, which groups are unsubstituted or substituted by one or more, same or different R^{e}; or
C₃-C₈-cycloalkyl, C₃-C₈-cycloalkenyl, heterocyclyl, aryl, hetaryl, C₃-C₈-cycloalkyl-C₁-C₆-alkyl, C₃-C₈-cycloalkenyl-C₁-C₆-alkyl, heterocyclyl-C₁-C₆-alkyl, aryl-C₁-C₆-alkyl, hetaryl-C₁-C₆-alkyl, phenoxy, benzyloxy, wherein the cyclic moieties are unsubstituted or substituted by one or more, same or different R^{a};
R^{a} CN, halogen, NO₂, C₁-C₄-alkyl, C₁-C₄-haloalkyl, or C₁-C₄-alkoxy;
or two substituents R^{a} on adjacent C-atoms form a bridge CH₂CH₂CH₂CH₂, OCH₂CH₂CH₂, CH₂OCH₂CH₂, OCH₂CH₂O, OCH₂OCH₂, CH₂CH₂CH₂, CH₂CH₂O, CH₂OCH₂, O(CH₂)O, SCH₂CH₂CH₂, CH₂SCH₂CH₂, SCH₂CH₂S, SCH₂SCH₂, CH₂CH₂S, CH₂SCH₂, S(CH₂)S, and form together with the C atoms, to which the two R^{a} are bonded to, a 5-membered or 6-membered saturated carbocyclic or heteocyclic ring;
R^{b} H, C₁-C₆-alkyl, C₂-C₄-alkenyl, C₂-C₄-alkynyl, C₁-C₄-haloalkyl, phenyl or benzyl;
R^{c}, R^{d} are independently of each other H, C₁-C₄-alkyl, or C₁-C₄-haloalkyl; or
R^{c} and R^{d} together with the N atom to which they are bonded form a 5- or 6-membered, saturated or unsaturated heterocycle, wherein the heterocycle is unsubstituted or substituted with one or more, same or different halogen atoms;
R^{e} CN, halogen, C₁-C₄-alkyl, C₁-C₄-haloalkyl, C₁-C₄-alkoxy, or C₁-C₄-haloalkoxy;
Y O or S; and
m 0, 1 or 2.

2. The composition according to claim 1, wherein the variables of the compound of formula (I) have the following meaning:
R^{a} halogen, C₁-C₂-alkyl, C₁-C₂-alkoxy;
or two substituents R^{a} on adjacent C-atoms are a OCH₂CH₂O bridge or a O(CH₂)O bridge;
R^{b} H, C₁-C₆-alkyl, phenyl and benzyl;
R^{c}, R^{d} are independently H, C₁-C₄-alkyl, or C₁-C₄-haloalkyl; and
R^{e} halogen and C₁-C₄-alkyl.

3. The composition according to claim 1, wherein the variables of the compound of formula (I) have the following meaning:
R¹, R² are independently H, C₂-C₆-alkynyl, C₂-C₆-alkynyloxy, aryl-C₁-C₆-alkyl, or hetaryl-C₁-C₆-alkyl;
wherein least one of R¹ and R² is H.

4. The composition according to claim 1, wherein the variables of the compound of formula (I) have the following meaning:
A phenyl, which is unsubstituted or one or more, same or different R^{A}; and
R^{A} halogen, NO₂, NR^{c}R^{d}, C₁-C₆-alkyl, C₁-C₆-haloalkyl, C₁-C₆-alkoxy, C₁-C₆-alkylthio, phenoxy, or benzyloxy, wherein the cyclic moieties are unsubstituted or substituted with one or more, same or different R^{a}.

5. The composition according to claim 1, wherein the variables of the compound of formula (I) have the following meaning:
R¹, R² are H; and
A is phenyl, which is substituted with Cl.

6. The composition according to claim 1, wherein the weight ratio of the compound of formula (I) to ZIF-8 is from 1:10 to 2:1.

7. The composition according to claim 1 comprising a fertilizer.

8. The composition according to claim 7, wherein the fertilizer is an organic, or inorganic ammonium-containing fertilizer, or a urea-containing fertilizer.

9. A method for fertilization, comprising treating plant propagation material, a plant growing on soil or soil substituents and/or the locus or soil or soil substituents where the plant is growing or is intended to grow with the composition as defined in claim 1.

10. The method according to claim 10, wherein the plant propagation material, the plant and/or the locus or soil or soil substituents where the plant is growing or is intended to grow is additionally treated with a fertilizer.

11. A method for reducing the evaporation rate of the compound of formula (I) comprising the use of ZIF-8.

12. A method for producing the composition as defined in claim 1 comprising a step a) of adsorbing the compound of formula (I) on the metal-organic-framework ZIF-8.

13. The method according to claim 12 comprising a step b) of co-granulating the compound of formula (I) adsorbed on ZIF-8 with a fertilizer, wherein the composition is in the form of granules.

14. The method according to claim 13, wherein the temperature in step b) is from 50° C. to 150° C.

15. (canceled)
